(19) United States
(12) Patent Application Publication
(10) Pub. No.: US 2023/0017588 A1
(43) Pub. Date: Jan. 19, 2023

(54) LOCAL WEARABLE BRAIN WAVE CAP DEVICE FOR DETECTION

(71) Applicant: ExeBrain Co., Ltd., Taipei (TW)

(72) Inventors: Shu-Chun Kuan, Taipei City (TW); Jason Chun-Cheng Lin, Taipei City (TW); Chung-Tse Shen, Taipei City (TW)

(21) Appl. No.: 17/494,865

(22) Filed: Oct. 6, 2021

(30) Foreign Application Priority Data
Jul. 13, 2021 (TW) 110125765

(51) Int. Cl. A61B 5/374; A61B 5/00; A61B 5/024; A61B 5/291; A61B 5/31; A61B 5/384

(52) U.S. Cl. CPC A61B 5/374; A61B 5/0006; A61B 5/02405; A61B 5/291; A61B 5/31; A61B 5/384; A61B 5/725; A61B 5/6803

(57) ABSTRACT

A local wearable brain wave cap device for detection is provided to simultaneously detect brainwave and heart rate variability data of a subject and includes a brain wave detection cap, at least one ear electrode and a transmission unit. The brain wave detection cap includes a wearable device and a plurality of electrode units. The wearable device is suitable for arranging the plurality of electrode units on brain wave positions corresponding to head of a subject. Each of the plurality of electrode units includes an accelerator, a storage unit, an input/output unit and a primary amplifier for detecting a brain wave.

### CROSS-REFERENCE TO RELATED APPLICATION

This application claims the priority of Taiwanese patent application No. 110125765, filed on Jul. 13, 2021, which is incorporated herewith by reference.

### BACKGROUND OF THE INVENTION

### 1. Field of the Invention

The present invention relates to a brain wave cap device, and particularly to a local wearable brain wave cap device for detection.

### 2. The Prior Arts

In the existing biological feedback training, the wireless devices at the input is mainly used, such as for comparing brain wave changes before and after training with a pair of electrode patches for the three areas of the parietal lobe, and detecting influence of neurophysiological feedback to the sensorimotor rhythm (SMR) with a pair of electrode patches, or for collecting physiological signals, and uploading physiological data to cloud platform via wired or wireless transmission modules for analysis. A user needs to open APP or related applications to read the physiological device worn during sleep in retrospect. However, the prior art usually prevents the user from obtaining physiological information such as brain waves or heart rate variability immediately, and the user needs to wait several hours to several days for interpretation.

Therefore, it is needed to provide improved methods and systems that can provide real-time feedback remotely and allow the user to immediately understand conditions, and to adjust physiological signals through visual or audial feedback for recovery.

### SUMMARY OF THE INVENTION

To solve the problems mentioned above, the present invention provides a local wearable brain wave cap device for detection to simultaneously detect brain wave and heart rate variability data of a subject, which includes a brain wave detection cap, at least one ear electrode and a transmission unit. The brain wave detection cap includes a wearable device and a plurality of electrode units. The wearable device is suitable for arranging the plurality of electrode units on brain wave positions corresponding to head of a subject. Each of the plurality of electrode units includes an accelerator, a storage unit, an input/output unit and a primary amplifier for detecting a brain wave. The primary amplifier is connected to the input/output unit and outputs a brain wave signal. The at least one ear electrode worn on an ear of the subject, which is used for detection to output a pulse signal. The transmission unit electrically connected to the plurality of electrode units and the ear electrode includes a secondary amplifier, a processing unit and a signal transmission unit. The secondary amplifier amplifies a brain wave signal output by the plurality of electrode units and the pulse signal output by the ear electrode. The processing unit encodes the brain wave signal corresponding to the brain wave positions and the pulse signal. The signal transmission unit outputs the brain wave signal and the pulse signal after being encoded.

According to an embodiment of the present invention, the transmission unit is a wireless transmission unit, and the wireless transmission unit transmits the brain wave signal and the pulse signal amplified by the secondary amplifier to a dongle to make a docking device installed with the dongle to process the brain wave signal and the pulse signal.

According to an embodiment of the present invention, the transmission unit is a wired transmission unit, and the wired transmission unit is electrically connected to a docking device to transmit the brain wave signal and the pulse signal amplified by the secondary amplifier to the docking device, and the docking device processes the brain wave signal and the pulse signal.

According to an embodiment of the present invention, the pulse signal is used to analyze a heart rate variability, an autonomic nerve balance, and a brain function.

According to an embodiment of the present invention, types of the brain wave signals that can be detected by the plurality of electrode units include 19, 32, or 64 channels.

According to an embodiment of the present invention, the brain wave signal is converted by frequency spectrum algorithm to obtain amplitude, frequency, and brain wave characteristics of the corresponding brain wave position.

According to an embodiment of the present invention, the plurality of electrode units detect the frequency, amplitude, coherence, phase lag and brainwave frequency band ratio.

According to an embodiment of the present invention, the wearable device of the brain wave detection cap is worn on nasion and inion of the subject.

According to an embodiment of the present invention, the local wearable brain wave cap for detection is connected to a docking device via the signal transmission unit, and the docking device is used for uploading the brain wave signal and the pulse signal to a cloud server for comparison.

The present invention further provides a method for assessing cardiovascular age and detecting brain age, which includes: using a plurality of electrodes to receive brain wave of a subject; using a primary amplifier to amplify the brain wave being received; transmitting the amplified brain wave through a shielding isolation circuit to a filter for filtering; and transmitting output of the filter to a secondary amplifier to transmit the brain wave and the heart rate variability to a signal transmission transmitter.

The local wearable brain wave cap device of the present invention can analyze signal more deeply, and can simultaneously increase the evaluation efficiency, the spatial resolution of collected brain wave, the detection range and sensitivity of brain wave analysis. As for use in the biological feedback training system, real-time feedback can be achieved at the remote end, which allows the user to immediately understand conditions, and to adjust physiological signals through visual or audial feedback for recovery.

### BRIEF DESCRIPTION OF THE DRAWINGS

FIG. 1 is a schematic diagram of a system for biological feedback training according to an embodiment of the present invention;

FIG. 2 is a top view of the embodiment of FIG. 1 in use;

FIG. 3 is a schematic diagram of an ear electrode according to an embodiment of the present invention;

FIG. 4 is a schematic diagram of the local wearable brain wave cap for detection in FIG. 1;

FIG. 5 is a schematic diagram of using the local wearable brain wave cap for detection according to an embodiment of the present invention;

FIG. 6 is a schematic diagram of using the local wearable brain wave cap for detection according to another embodiment of the present invention;

FIG. 7 is another schematic diagram of the local wearable brain wave cap for detection in FIG. 1;

FIG. 8 is a schematic diagram of an electrode structure of the local wearable brain wave cap for detection in FIG. 1; and

FIG. 9 is a schematic diagram of signal transmission of the local wearable brain wave cap for detection in FIG. 1.

### DETAILED DESCRIPTION OF THE PREFERRED EMBODIMENT

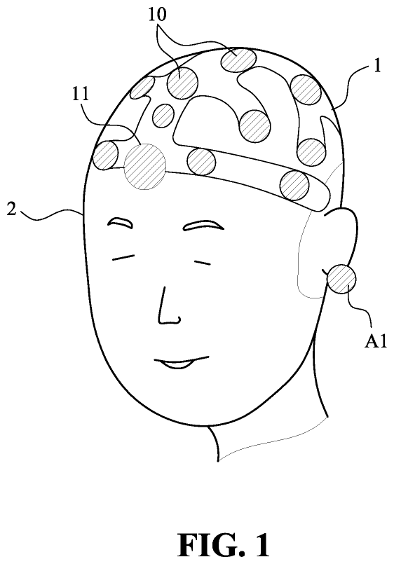

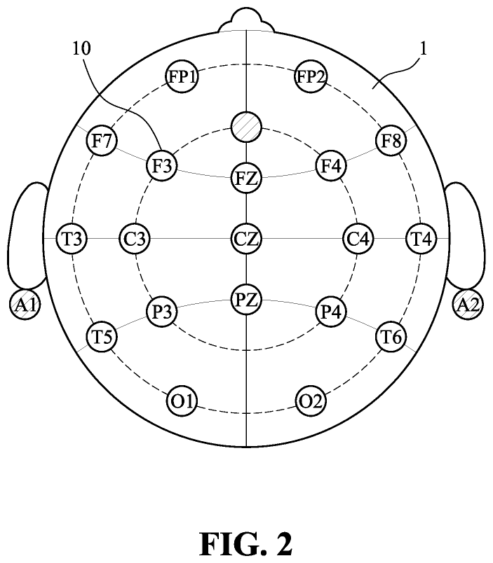

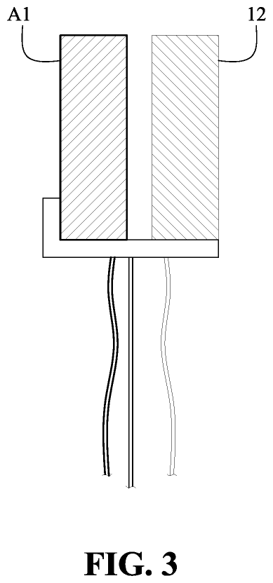

Please refer to FIG. 1, FIG. 1 is a schematic diagram of a system for biological feedback training according to an embodiment of the present invention. The local wearable brain wave cap device for detection used in the biological feedback training system of the present invention includes a local wearable brain wave cap 1 for detection, which includes a wearable device worn on nasion and inion of the subject, and equipped with a plurality of electrode units 10 on the brain wave positions corresponding to the head of the subject, such as the electrode units FP1, FP2, F3, F4, F7, F8, FZ, T3, C3, CZ, C4, T4, T5, P3, PZ, P4, T6, 01, and 02 shown in the top view of FIG. 2 are used to detect a brain wave of the subject 2. The plurality of electrode units may include gel electrodes and dry electrodes. The plurality of electrodes can detect different frequency, amplitude, coherence, phase lag and ratio of frequency band of brain waves with frequency of 1-100 Hz. The local wearable brain wave cap 1 for detection needs to be a daily, weekly, monthly, or bimonthly disposable. As shown in the top view of FIG. 2, the local wearable brain wave cap device for detection includes two ear electrodes A1 and A2, which are worn on the subject's ears for detecting a heart rate variability (HRV), an autonomic nerve balance condition and a brain function to output a pulse signal, which is used to analyze a heart rate variability, an autonomic nerve balance condition and a brain function. It can be seen from FIG. 3 that, taking the ear electrode A1 as an example, the ear electrode is individually connected to a second ear electrode 12 for transmitting blood flow and heart rate variability data for analysis of autonomic nerve function.

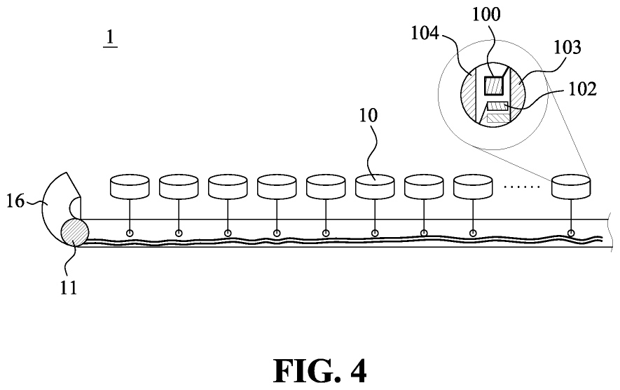

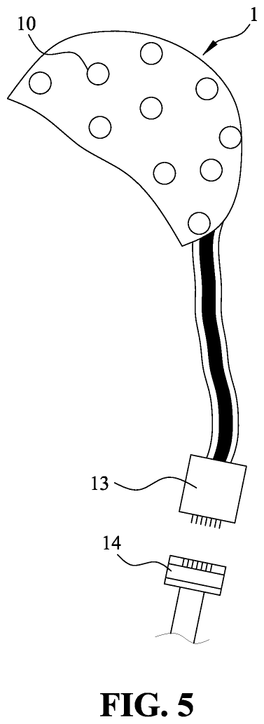

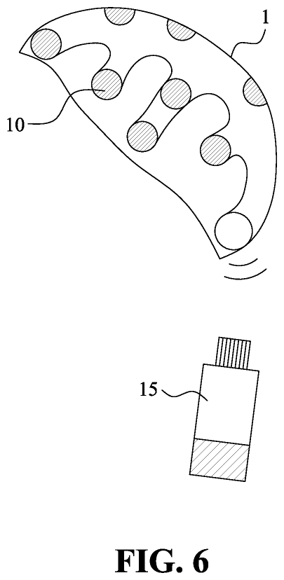

Please refer to FIG. 4, the local wearable brain wave cap 1 for detection includes a transmission unit 19 electrically connected to the electrode units FP1, FP2, F3, F4, F7, F8, FZ, T3, C3, CZ, C4, T4, T5, P3, PZ, P4, T6, 01, 02 and the ear electrodes A1 and A2, and includes a primary amplifier 11, a processing unit, and a signal transmission unit. The secondary amplifier 11 amplifies the brain wave signal output by the electrode units FP1, FP2, F3, F4, F7, F8, FZ, T3, C3, CZ, C4, T4, T5, P3, PZ, P4, T6, 01, 02 and the pulse signal output by the ear electrodes A1 and A2. The processing unit encodes the plurality of brain wave signals corresponding to the brain wave positions and the pulse signals. The brain wave signal is converted by frequency spectrum algorithm to obtain the amplitude, frequency and brain wave characteristics of the corresponding brain wave position. The signal transmission unit is used for outputting the encoded brain wave signal and pulse signal. Each electrode unit 10 has an input and output unit 100, a primary amplifier 102, a storage unit 103, and an accelerator 104. The transmission unit may be the wired transmission unit of FIG. 5 or the wireless transmission unit of FIG. 6, as shown in FIG. 5, the wired transmission unit is electrically connected to the docking device through the wired connector 13 and the adapter 14 so that the brain wave signal and the pulse signal amplified by the amplifier 11 are transmitted to the docking device, and the docking device processes the brain wave signal and the pulse signal. As shown in FIG. 6, the wireless transmission unit transmits the brain wave signal and the pulse signal amplified by the secondary amplifier 11 to the dongle 15 so that the local wearable brain wave cap 1 for detection can perform remote transmission.

The biological feedback training system proposed by the present invention uses the local wearable brain wave cap 1 for detection, and includes a docking device, a dongle, and a remote real-time feedback training module. The local wearable brain wave cap 1 for detection is used to simultaneously detect the brain wave and heart rate variability data of the subject 2 to obtain a biological indicator, and to detect brain age and cardiovascular age for a stroke or brain functional risk prediction. The docking device which can be a computer/mobile phone, etc., is wirelessly or wiredly connected to the portable brain wave cap 1 for detection through the dongle 15 or the wired connector 13 and the adapter 14 to expand the memory and the calculation speed to compare a biological indicator with a detection database containing the brain wave and the heart rate variability data to generate a comparison result. The comparison of the detection database can be performed based on the health norm of the healthy person and the clinical norm of the disease by the calculation of the communication media. The remote real-time feedback training module receives the comparison result, and performs a real-time biological feedback training through a remote transmission according to the comparison result.

In this embodiment, the real-time biological feedback training uses 19 channels (that is, the above-mentioned electrodes FP1, FP2, F3, F4, F7, F8, FZ, T3, C3, CZ, C4, T4, T5, P3, PZ, P4, T6, 01, 02) for neurophysiological feedback training, in addition to training the surface cortex of the brain, it can also train the deep cortex of the brain and train specific brain regions. It can also collect the current changes of the frontal, parietal, temporal and occipital lobes of the human brain. It should be noted that in other embodiments, the real-time biological feedback training uses 32 or 64 channels. In this embodiment, the neurophysiological feedback training uses electroencephalogram (EEG) and 19 channels to stereolocate a specific region of the brain to be trained.

In addition, compared with fixed hard cap of the prior art, the brain wave cap used in the present invention uses a soft disposable material, which can be worn directly, or the user can install the brain wave sensor in an easily equipped wearable a cap, such as a helmet, which can detect brain wave signals at 19, 32, or 64 channels. The current changes that can be collected include the frontal, parietal, temporal and occipital lobes of the human brain, the current changes on the frontal leaf of the human body is mainly detected. The channel positions are the positions of the general brain wave cap. The channel position of the prior art has to be injected with conductive gel (or called “gel-electrode”). The present invention mainly emphasizes the convenience of wearing, and the novel electrode is used to collect the brain wave signal, and the fixed channel position in the present invention conforms to the international standard positioning specification “International 10-20 system”.

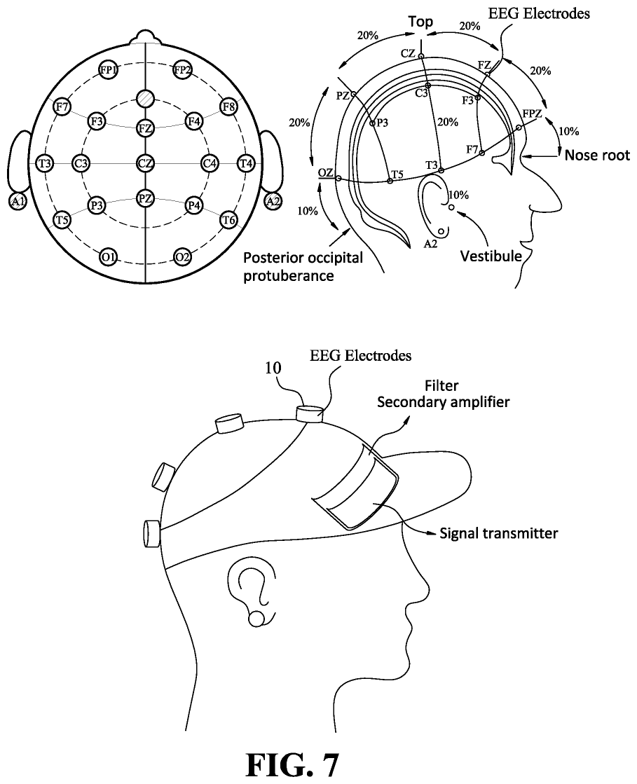

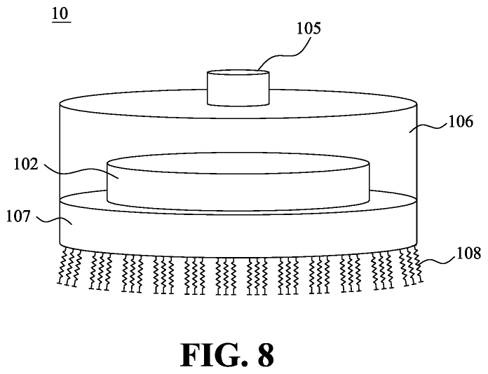

The present invention mainly uses a printed circuit board (PCB) as the base cap material and has four types: single use disposable, weekly disposable, monthly disposable, and bi-monthly disposable, the four types of electrodes used to collect brain waves can be gel electrodes (gel liquid electrolyte used to increase conductivity) or dry electrodes. The electrodes can be replaced directly by snapping. In addition, please refer to FIGS. 7 and 8. FIG. 7 is another schematic diagram of the local wearable brain wave cap for detection in FIG. 1, and FIG. 8 is a schematic diagram of the electrode structure of the local wearable brain wave cap for detection in FIG. 1. FIG. 7 shows the filter 109, the secondary amplifier 11 and the signal transmitter 110 of the local wearable brainwave cap for detection, and the position of the head wearable device and the distributed positions of the electrodes 10. As shown in FIG. 8, the electrode 10 includes a primary amplifier 102, a connecting buckle 105, a shielding isolation circuit 106, a connector 107, and a spring brush electrode 108. Because the contact point of the spring brush electrode 108 has a shape of a spring brush, which can directly contact the scalp in a non-invasive and painless manner to collect signals more clearly.

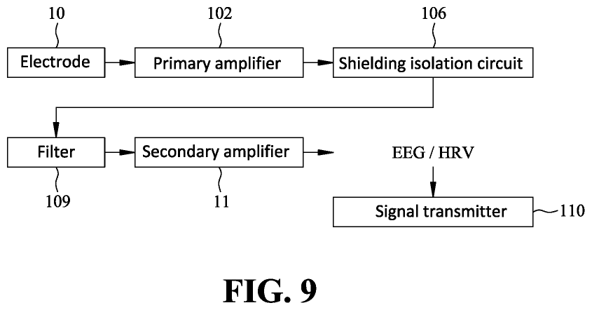

Furthermore, the brain age of the subject can be analyzed through the brain wave cap, and the brain age is divided into overall brain age, frontal lobe brain age, parietal lobe brain age, occipital lobe brain age, and temporal lobe brain age. In addition, the age of each network of the brain can also be analyzed. Finally, the ear electrode detects the HRV through the photoplethysmography (PPG) method or detects the ECG/EKG waveform through the ear blood vessels. The principle is to illuminate the LED light source and then receive the detected light intensity on the other side, the received detected light intensity will change due to the change in the volume of the end caused by the heart rate. Through this change, we can analyze the cardiovascular age of the subject. The PPG signal received by the ear electrode can be converted into HRV and the cardiovascular age can also be analyzed to assess the cardiovascular age and perform brain age detection as a risk predictor for stroke and cardiovascular disease. The above-mentioned signal transmission process is shown in FIG. 9, the brain wave EEG and the heart rate variability HRV are transmitted to the signal transmitter 110 through the electrode 10, the primary amplifier 102, the shielding isolation circuit 106, the filter 109, and the secondary amplifier 11 in sequence.

The 19 channel positions of the present invention can analyze different frequency, amplitude, coherence, phase lag and ratio of frequency band of brain waves with frequency of 1-100 Hz, so as to get deeper analysis of the signal and more efficient evaluation. In addition, the brain wave cap and the ear electrode of the present invention record brain waves and heart rate variability, respectively, and also analyze the balance of autonomic nerves and the activity of brain functions.

Compared with a brain wave collection device that has only 6 channels, the present invention can be a brain wave collection device using 19, 32, or 64 channel positions, so that the spatial resolution of brain wave collection can be increased. In addition, the lowest sampling frequency of the present invention is above 250 Hz, so the detection range for brain wave analysis is larger and the sensitivity is higher.

The amplifier in the brain wave cap of the present invention is externally connected to the back of the head, and the brain waves collected in each recording channel need to be transmitted to the amplifier through a wired line. The invention mainly collects brain waves through the printed circuit board, and the primary and secondary amplifiers are respectively placed at each recording position and the brain wave cap edge.

The present invention also improves the positioning efficiency. If the traditional brain wave cap has deviations in the position of each electrode, there may be a gap in data analysis. Therefore, the brain wave cap of the present invention is particularly in contact with the nasion and the inion to reduce the volume of the brainwave cap amplifier without reducing the signal quality, and thus not susceptible to interference from other external electrical signals. Compared with traditional devices which has no feedback function, the present invention can not only collect brain wave and heart rate variability parameters, but also can analyze the interactive relationship between autonomic nerves and brain nerves in real time.

In summary, the biological feedback training method proposed by the present invention includes three stages: signal transmission (the subject 2), real-time calculation (the docking device), signal feedback (remote real-time feedback training module). The above three stages include features such as immediacy, accuracy and immunity from other noise interference. In some embodiments, in addition to a computer, the docking device also includes screen synchronous control, so that the remote real-time feedback training module can use neural feedback software to adjust the training parameters of the subject 2. In addition, when the docking device is used with a mobile phone for training, due to the limited capacity of the mobile phone's memory, in some embodiments, the docking device can be used with the mobile phone's external computing software and hardware devices, so that the subject 2 can receive neurophysiological feedback training through the mobile phone.

The system and method for biological feedback training with a local wearable brain wave cap device for detection provided by the present invention can provide real-time (within one minute) feedback remotely, and allow the subject to immediately understand the conditions, and the subject can adjust the physiological signals for recovery through visual or audial feedback. The 19 channel positions enable deeper analysis of the signals and improve the efficiency of evaluation, the spatial resolution of brainwave collection, and detection range and sensitivity of the analysis of brain waves.

The present invention is not limited to the above-mentioned embodiments. It is obvious to those skilled in the art that various modifications and changes can be made to the present invention without departing from the spirit or scope of the present invention.

Therefore, the present invention is intended to cover the modifications and changes made to the present invention or falling within the scope of the claims and its equivalent scope.

What is claimed is:

1. A local wearable brain wave cap device for detection, which simultaneously detects brain wave and heart rate variability data of a subject and comprises:
a brain wave detection cap including a wearable device and a plurality of electrode units, wherein the wearable device suitable for arranging the plurality of electrode units on brain wave positions corresponding to head of a subject, each of the plurality of electrode units includes an accelerator, a storage unit, an input/output unit and a primary amplifier for detecting a brain wave;
at least one ear electrode worn on an ear of the subject, which is used for detection to output a pulse signal; and
a transmission unit electrically connected to the plurality of electrode units and the ear electrode, which includes:
a secondary amplifier amplifying a brain wave signal output by the electrode unit and the pulse signal output by the ear electrode;
a processing unit encoding the brain wave signal corresponding to the brain wave positions and the pulse signal; and
a signal transmission unit outputting the brain wave signal and the pulse signal after being encoded.

2. The local wearable brain wave cap device for detection of claim 1, wherein the transmission unit is a wireless transmission unit, and the wireless transmission unit transmits the brain wave signal and the pulse signal amplified by the secondary amplifier to a dongle to make a docking device installed with the dongle to process the brain wave signal and the pulse signal.

3. The local wearable brain wave cap device for detection of claim 1, wherein the transmission unit is a wired transmission unit, and the wired transmission unit is electrically connected to a docking device to transmit the brain wave signal and the pulse signal amplified by the secondary amplifier to the docking device, and the docking device processes the brain wave signal and the pulse signal.

4. The local wearable brain wave cap device for detection of claim 1, wherein the pulse signal is used to analyze a heart rate variability, an autonomic nerve balance, and a brain function.

5. The local wearable brain wave cap device for detection of claim 1, wherein types of the brain wave signals that can be detected by the plurality of electrode units include 19, 32, or 64 channels.

6. The local wearable brain wave cap device for detection of claim 1, wherein the brain wave signal is converted by frequency spectrum algorithm to obtain amplitude, frequency, and brain wave characteristics of the corresponding brain wave position.

7. The local wearable brain wave cap device for detection of claim 1, wherein the plurality of electrode units detect the frequency, amplitude, coherence, phase lag and brainwave frequency band ratio.

8. The local wearable brain wave cap device for detection of claim 1, wherein the wearable device of the brain wave detection cap is worn on nasion and inion of the subject.

9. The local wearable brain wave cap device for detection of claim 1, wherein the local wearable brain wave cap for detection is connected to a docking device via the signal transmission unit, and the docking device is used for uploading the brain wave signal and the pulse signal to a cloud server for comparison.

10. A method for assessing cardiovascular age and detecting brain age, which comprises:
using a plurality of electrodes to receive brain wave of a subject;
using a primary amplifier to amplify the brain wave being received;
transmitting the amplified brain wave through a shielding isolation circuit to a filter for filtering; and
transmitting output of the filter to a secondary amplifier to transmit the brain wave and the heart rate variability to a signal transmission transmitter.
